(19) United States
(12) Patent Application Publication
(10) Pub. No.: US 2026/0110008 A1
(43) Pub. Date: Apr. 23, 2026

(54) MUTATED LACTO-N-BIOSIDASE

(71) Applicant: DSM IP Assets B.V., Maastricht (NL)

(72) Inventors: Birgitte ZEUNER, Søborg (DK); Jan MUSCHIOL, Osdorf (DE); Marlene Isabelle VUILLEMIN, Søborg (DK); Anne Merete Boye Strunge MEYER, København C (DK); Jesper HOLCK, Hvidovre (DK)

(21) Appl. No.: 19/138,531

(22) PCT Filed: Dec. 14, 2023
(86) PCT No.: PCT/EP2023/085799

§ 371 Date: Jun. 12, 2025

(30) Foreign Application Priority Data
Dec. 14, 2022 (DK) PA202201151

(51) Int. Cl. C12P 19/26; C12N 9/24

(52) U.S. Cl. CPC C12P 19/26; C12N 9/2402; C12Y 302/0114

(57) ABSTRACT

Mutated lacto-N-biosidases are provided with improved properties compared to the wild type lacto-N-biosidase isolated from Bifidobacterium longum.

### FIELD OF THE INVENTION

This invention relates to a lacto-N-biosidase having increased transglycosidase synthesis activity and enzymatic reactions to make oligosaccharides with a lacto-N-biose terminal at the non-reducing end in one enzymatic step.

### BACKGROUND OF THE INVENTION

During the past decades, the interest in the preparation and commercialisation of human milk oligosaccharides (HMOs) has been increasing steadily. The importance of HMOs is directly linked to their unique biological activities, therefore HMOs have become important potential products for nutrition and therapeutic uses. As a result, low cost ways of producing industrially HMOs have been sought.

To date, the structures of more than 140 HMOs have been determined, and considerably more are probably present in human milk. The HMOs comprise a lactose (Galβ1-4Glc) moiety at the reducing end and may be elongated with an N-acetylglucosamine, or one or more N-acetyllactosamine moiety/moieties (Galβ1-4GlcNAc) and/or a lacto-N-biose moiety (Galβ1-3GlcNAc). Lactose and the N-acetyllactosaminylated or lacto-N-biosylated lactose derivatives may further be substituted with one or more fucose and/or sialic acid residue(s), or lactose may be substituted with an additional galactose, to give HMOs known so far. In human milk/colostrum, type I oligosaccharides containing lacto-N-biose predominate over type II oligosaccharides that contain N-acetyllactosamine. This is a human specific feature which is not found in other mammals. The predominance of type I oligosaccharides is of interest in relation to the formation of the bifidus flora in the infant colon and may potentially have other benefits. Whereas N-acetyllactosamine can serve as both the internal and non-reducing end terminal disaccharide unit(s), lacto-N-biose can only serve as the non-reducing end terminal disaccharide (Urashima et al.: Milk oligosaccharides, Nova Biomedical Books, 2011; Chen Adv. Carbohydr. Chem. Biochem. 72, 113 (2015)). Core HMO structures with terminal lacto-N-biose are listed in Table 1 below.

TABLE 1

| core name | core structure |
|---|---|
| lacto-N-tetraose (LNT) | Galβ1-3GlcNAcβ1-3Galβ1-4Glc |
| lacto-N-hexaose (LNH) | Galβ1-3GlcNAcβ1-3(Galβ1-4GlcNAcβ1-6)Galβ1-4Glc |
| para-lacto-N-hexaose (pLNH) | Galβ1-3GlcNAcβ1-3Galβ1-4GlcNAcβ1-3Galβ1-4Glc |
| lacto-N-octaose (LNO) | Galβ1-3GlcNAcβ1-3(Galβ1-4GlcNAcβ1-3Galβ1-4GlcNAcβ1- |
| | 6)Galβ1-4Glc |
| lacto-N-neooctaose (LNnO) | Galβ1-4GlcNAcβ1-3(Galβ1-3GlcNAcβ1-3Galβ1-4GlcNAcβ1- |
| | 6)Galβ1-4Glc |
| iso-lacto-N-octaose (iLNO) | Galβ1-3GlcNAcβ1-3(Galβ1-3GlcNAcβ1-3Galβ1-4GlcNAcβ1- |
| | 6)Galβ1-4Glc |
| para-lacto-N-octaose (pLNO) | Galβ1-3GlcNAcβ1-3Galβ1-4GlcNAcβ1-3Galβ1-4GlcNAcβ1- |
| | 3Galβ1-4Glc |
| lacto-N-neodecaose (LNnD) | Galβ1-3GlcNAcβ1-3[Galβ1-4GlcNAcβ1-3(Galβ1-4GlcNAcβ1- |
| | 6)Galβ1-4GlcNAcβ1-6]Galβ1-4Glc |
| lacto-N-decaose (LND) | Galβ1-3GlcNAcβ1-3[Galβ1-3GlcNAcβ1-3(Galβ1-4GlcNAcβ1- |
| | 6)Galβ1-4GlcNAcβ1-6]Galβ1-4Glc |

Efforts to develop processes for synthesizing HMOs have increased significantly in the last ten years. In this regard, processes have been developed for producing them by microbial fermentations, enzymatic processes, chemical syntheses, or combinations of these technologies. Although the synthesis and purification of HMOs with simpler structure, for example tri- or tetrasaccharide HMOs, in industrial scale, has recently been accomplished by multiple manufacturers using biotechnological methods comprising the utilization of genetically modified microorganisms, the same task for HMOs with more complicated structures is still challenging.

In biological systems, Leloir-type glycosyltransferases (GTs, EC 2.4.1. —) and glycosidases (also called glycoside hydrolases: GHs, EC 3.2.1. —) constitute the two major classes of carbohydrate-processing enzymes, which may be utilized in the production of HMOs. Both classes of enzymes act to transfer a glycosyl group from a donor to an acceptor resulting in oligosaccharide production. The use of glycosyltransferases for synthesis in industrial in vitro processes is limited mainly by the high costs of the activated donor sugars. In contrast to glycosyltransferases, glycosidases have a wide range of donor substrates employing usually monosaccharides, oligosaccharides or/and engineered substrates (i.e. substrates carrying various functional groups). They often display activity towards a large variety of carbohydrate and non-carbohydrate acceptors. Another advantage of the use of glycosidases in vitro compared to glycosyltransferases is their robustness and accessibility.

Lacto-N-biosidases (EC 3.2.1.140) are mostly classified in the GH20 family. Lacto-N-biosidases typically proceed through a retaining mechanism and specifically hydrolyse the terminal lacto-N-biosyl residue from the non-reducing end of oligosaccharides. Lacto-N-biosidase from Bifidobacterium bifidum JCM1254 (LnbB) was shown to have a low transglycosylation activity to make LNT from suitable precursors (Wada et al. Appl. Environ. Microbiol. 74, 3996 (2008)), and other lacto-N-biosidases have also been suggested for enzymatic HMO synthesis (WO 2012/156897, WO 2012/156898). Recently, specific mutants of the lacto-N-biosidase LnbB with reduced hydrolytic activity have been disclosed that were able to produce LNT from suitable lacto-N-biose donors and lactose (WO 2020/126613, Castejón-Vilatersana et al. Int. J. Mol. Sci. 22, 3230 (2021), Vuillemin et al. Appl. Sci. 11, 11493 (2021)).

Sakamura et al. (J. Biol. Chem. 288, 25194 (2013)) revealed a lacto-N-biosidase from B. longum JCM1217 (LnbX) that completely differs from hitherto identified GH20 lacto-N-biosidases with respect to amino acid sequence, substrate specificity, structure and catalytic mechanism. LnbX is the founding member of the GH136 family.

New solutions have been sought for the enzymatic synthesis of type I oligosaccharides, preferably type I HMOs, especially type I HMOs beyond LNT.

### SUMMARY OF THE INVENTION

The present invention relates to a mutated lacto-N-biosidase having
an amino acid sequence that is substantially identical, that is having at least 70% sequence identity, to the sequence from amino acid position 45 to 625 of SEQ ID No. 1, and
a mutation at least at one or more of amino acid positions selected from 410, 416, 439 and 442, said amino acid numbering being according to SEQ ID No. 1.

Preferably, the mutated lacto-N-biosidase comprises one or more of the following mutations:
at position 410 Gly (G) is substituted by Trp, Tyr, Phe or His, preferably Trp; and/or
at position 416 Asp (D) is substituted by Asn or Gln, preferably Asn; and/or
at position 439 Met (M) is substituted by Leu, Val or lie, preferably Leu, and/or
at position 442 Asn (N) is substituted by Trp, Tyr, Phe or His, preferably Trp.

In one embodiment, the mutated lacto-N-biosidase has an amino acid sequence that is substantially identical to the sequence from amino acid position 31 to 625 of SEQ ID No. 1, and a mutation at least at one or more of amino acid positions selected from 410, 416, 439 and 442, said amino acid numbering being according to SEQ ID No. 1.

According to another aspect, the invention relates to a process for making a mutated lacto-N-biosidase mentioned above comprising the steps of:
(a) providing a DNA sequence encoding the mutated lacto-N-biosidase, then
(b) expressing the mutated lacto-N-biosidase in a host cell transformed with the DNA sequence obtained in step (a).

Also, a method for synthesizing a lacto-N-biose containing carbohydrate is provided comprising the step of reacting a lacto-N-biosyl donor and a carbohydrate acceptor in the presence of a lacto-N-biosidase comprising an amino acid sequence that is substantially identical, that is having at least 70% sequence identity, to the sequence from amino acid position 31 or 45 to 625 of SEQ ID No. 1 to transfer the lacto-N-biosyl residue of the donor to the carbohydrate acceptor.

In a further aspect of the invention, use of a lacto-N-biosidase comprising an amino acid sequence that is substantially identical, that is having at least 70% sequence identity, to the sequence from amino acid position 31 or 45 to 625 of SEQ ID No. 1 for the preparation of a lacto-N-biose containing carbohydrate is provided.

In a further aspect of the invention, it is provided a mixture consisting or consisting essentially of LNT, LNnT, pLNH and optionally lactose.

In a further aspect of the invention, it is provided a process for obtaining the mixture according to the fifth aspect of the invention, wherein said process comprises reacting LNT and LNnT in the presence of an enzyme selected from the group consisting of LnbX, its truncated functional analogs and a mutated lacto-N-biosidase according to the first aspect of the invention, to produce a reaction mixture, and then removing the enzyme and optionally lactose from the reaction mixture.

### DETAILED DESCRIPTION OF THE INVENTION

The present inventors surprisingly found that lacto-N-biosidase from B. longum JCM1217 (LnbX, Sakamura et al. J. Biol. Chem. 288, 25194 (2013), GenBank nr. DAA64542, SEQ ID No. 1) and its truncated functional analogs can be utilized as trans-lacto-N-biosidase to make linear lacto-N-biose containing oligosaccharides. Moreover, it was discovered that the yield of this enzymatic synthesis can be improved if LnbX or its truncated functional analogs are mutated at certain positions.

“Truncated functional analog” of LnbX means that the remaining truncated LnbX protein substantially retains the desired activity. Examples of truncated functional analogs of LnbX are polypeptide fragments from amino acid position 31 to 1573, 31 to 1431, 38 to 1431, 45 to 1431, 31 to 1005, 31 to 904, 31 to 639, 31 to 625 or 45 to 625 of SEQ ID No. 1, preferably 31 to 625 or 45 to 625.

The first aspect of the invention relates to a mutated lacto-N-biosidase comprising a polypeptide fragment having:
substantial identity (i.e. at least 70 percent (%) sequence identity) to a polypeptide fragment from amino acid position 45 to 625 of SEQ ID No. 1, and
mutation(s) (that is, an amino acid in the wild-type sequence replaced by another amino acid) at one or more amino acid positions selected from 410, 416, 439 and 442, said amino acid numbering being according to SEQ ID No. 1.
Preferably, the one or more amino acid positions are:
   position 410 wherein Gly (G) is substituted by Trp, Tyr, Phe or His, preferably Trp; and/or
   position 416 wherein Asp (D) is substituted by Asn or Gln, preferably Asn; and/or
   position 439 wherein Met (M) is substituted by Leu, Val or lie, preferably Leu, and/or
   position 442 wherein Asn (N) is substituted by Trp, Tyr, Phe or His, preferably Trp.

Thereby, a mutated lacto-N-biosidase can be obtained providing, in comparison with the wild-type lacto-N-biosidase of SEQ ID No. 1 or its truncated functional analogs such as an enzyme comprising the polypeptide fragment from amino acid position 31 or 45 to 625 of SEQ ID No. 1:
increased lacto-N-biosidase synthetic performance in a reaction between a lacto-N-biosyl donor and an acceptor to produce a lacto-N-biose containing product, and/or
reduced hydrolysis of the lacto-N-biosyl donor and/or the lacto-N-biose containing product.

Accordingly, the present invention provides a mutated lacto-N-biosidase comprising a polypeptide fragment having a sequence identity of at least 70% to a polypeptide fragment from amino acid position 45 to 625 of SEQ ID No.1, and
a) a mutation at one or more amino acid positions selected from 410, 416, 439 and 442, preferably wherein at least one of the mutations is selected from:
   at position 410 Gly (G) is substituted by Trp, Tyr, Phe or His, preferably Trp; and/or
   at position 416 Asp (D) is substituted by Asn or Gln, preferably Asn; and/or
   at position 439 Met (M) is substituted by Leu, Val or lie, preferably Leu, and/or
   at position 442 Asn (N) is substituted by Trp, Tyr, Phe or His, preferably Trp,
      and/or
b) increased trans-lacto-N-biosidase synthesis performance in a reaction between a lacto-N-biosyl donor and an acceptor to produce a lacto-N-biose containing product, and/or reduced hydrolytic activity towards the lacto-N-biose containing product of such a reaction, compared to the wild-type lacto-N-biosidase of SEQ ID No. 1 or its truncated functional analogs such as an enzyme comprising the polypeptide fragment from amino acid position 31 or 45 to 625 of SEQ ID No. 1.

The polypeptide fragment from amino acid position 31 to 625 of SEQ ID No.1 has been identified as the core part of LnbX responsible for full catalytic hydrolysis activity (Yamada et al. Cell Chem. Biol. 24, 515 (2017)). Furthermore, the polypeptide fragment from amino acid position 45 to 625 of SEQ ID No. 1 is identified as the relevant domain classifying LnbX in the GH136 family.

In accordance with this invention, the terms “substantial identity” and “substantially identical” in the context of two or more nucleic acid or amino acid sequences preferably mean that the two or more sequences are the same or have at least about 70% of amino acid residues that are the same when compared and aligned for maximum correspondence over a comparison window or designated sequences of nucleic acids or amino acids (i.e. the sequences have at least about 70 percent (%) identity). Percent identity of nucleic acid or amino acid sequences can be measured using a BLAST 2.0 (or higher) sequence comparison algorithms with default parameters, or by manual alignment and visual inspection (see e.g. http://www.ncbi.nlm.nih.gov/BLAST/). In accordance with this invention, the percent identity of substantially identical polypeptide fragment from amino acid position 31 or 45 to 625 of SEQ ID No.1, or substantially identical amino acid sequence of SEQ ID No. 1 is preferably at least 75%, more preferably at least 80%, even more preferably at least 85%, yet more preferably at least 90%, still even more preferably at least 92%, especially at least 93%, more especially at least 94%, even more especially at least 95%, yet even more especially at least 96%, particularly at least 97%, more particularly at least 98%, and most particularly at least 99%. Suitably, the definition preferably excludes 100% sequence identity, such as imposing a maximum limit on the sequence identity of 99.9%, 99.8%, or 99.7%, or requiring that at least one amino acid difference occurs between the sequences being compared. This definition also applies to the complement of a test sequence and to sequences that have deletions and/or additions, as well as those that have substitutions. An example of an algorithm that is suitable for determining percent identity and sequence similarity is the BLAST+2.13.0 algorithm, which is described in Altschul et al. Nucl. Acids Res. 25, 3389 (1997). BLAST+2.13.0 is used to determine percent sequence identity for the proteins of the invention. Software for performing BLAST analyses is publicly available through the National Center for Biotechnology Information (https://blast.ncbi.nlm.nih.gov/Blast.cgi).

LnbX transfers a lacto-N-biosyl residue from a donor substrate to an acceptor. If the acceptor is a carbohydrate (mono-, di- or oligosaccharide) then the lacto-N-biosidase acts as a trans-lacto-N-biosidase (able to make a lacto-N-biose containing carbohydrate product). On the other hand, the same lacto-N-biosidase can transfer the same lacto-N-biosyl residue, which was added to the carbohydrate acceptor previously, from the product to a water molecule, acting thus as a hydrolase. The two processes take place concurrently. The overall synthetic performance is the ratio of the trans-lacto-N-biosidase and hydrolysis activities.

By comparison, the mutant lacto-N-biosidases of this invention show a higher overall synthetic performance, which means a higher trans-lacto-N-biosidase activity relative to the hydrolytic activity. In this regard, a relatively low trans-lacto-N-biosidase synthetic activity of a mutant of this invention can be compensated by a significant reduction in the hydrolytic activity of the mutant that results in an improved synthetic performance. Similarly, a relatively high hydrolytic activity of a mutant can be overcome by a significant improvement in its trans-lacto-N-biosidase synthetic activity. The mutated lacto-N-biosidases of this invention show an improvement in their trans-lacto-N-biosidase synthetic performance over the wild-type LnbX or its truncated functional analogs.

Suitably, the mutant lacto-N-biosidases of the invention are non-natural lacto-N-biosidases, that is, they are not made in nature or naturally-occurring, but are made as a result of chemical synthesis, genetic engineering or similar methods in the laboratory, resulting in synthetic mutant lacto-N-biosidases.

Preferably, the lacto-N-biosidase of this first aspect comprises a polypeptide sequence that has a sequence identity of at least 70% to the segment from amino acid positions 45 to 625 of SEQ ID No.1 as described above, and the following amino acid mutations, in which:
at position 410 Gly (G) is substituted by Trp, Tyr, Phe or His, preferably Trp; and/or
at position 416 Asp (D) is substituted by Asn or Gln, preferably Asn.

Also preferably, the lacto-N-biosidase comprises a polypeptide sequence that has a sequence identity of at least 75%, preferably at least 80%, more preferably at least 85%, yet more preferably at least 90%, still even more preferably at least 95% to the segment from amino acid positions 45 to 625 of SEQ ID No.1 and a mutation:
at position 410 where Gly (G) is substituted by Trp, Tyr, Phe or His, preferably Trp; and/or
at position 416 where Asp (D) is substituted by Asn or Gln, preferably Asn; and/or
at position 439 where Met (M) is substituted by Leu, Val or lie, preferably Leu, and/or
at position 442 where Asn (N) is substituted by Trp, Tyr, Phe or His, preferably Trp.

Preferably, the lacto-N-biosidase of this first aspect comprises a polypeptide sequence that has a sequence identity of at least 75%, preferably at least 80%, more preferably at least 85%, yet more preferably at least 90%, still even more preferably at least 95% to the segment from amino acid positions 45 to 625 of SEQ ID No.1 as described above, and the following amino acid mutations, in which:
at position 410 Gly (G) is substituted by Trp, Tyr, Phe or His, preferably Trp; and/or
at position 416 Asp (D) is substituted by Asn or Gln, preferably Asn.

More preferably, the lacto-N-biosidase comprises a polypeptide sequence that is identical with the segment from amino acid positions 45 to 625 of SEQ ID No.1 and a mutation:
at position 410 where Gly (G) is substituted by Trp, Tyr, Phe or His, preferably Trp; and/or
at position 416 where Asp (D) is substituted by Asn or Gln, preferably Asn; and/or
at position 439 where Met (M) is substituted by Leu, Val or lie, preferably Leu, and/or
at position 442 where Asn (N) is substituted by Trp, Tyr, Phe or His, preferably Trp.

Even more preferably, the lacto-N-biosidase of this first aspect comprises a polypeptide sequence that identical with the segment from amino acid positions 45 to 625 of SEQ ID No.1, and the following amino acid mutations, in which:
at position 410 Gly (G) is substituted by Trp, Tyr, Phe or His, preferably Trp; and/or
at position 416 Asp (D) is substituted by Asn or Gln, preferably Asn.

In other preferred embodiment of the first aspect of the invention, the lacto-N-biosidase comprises a polypeptide sequence that has a sequence identity of at least 70% to the segment from amino acid positions 31 to 625 of SEQ ID No.1 and a mutation:
at position 410 where Gly (G) is substituted by Trp, Tyr, Phe or His, preferably Trp; and/or
at position 416 where Asp (D) is substituted by Asn or Gln, preferably Asn; and/or
at position 439 where Met (M) is substituted by Leu, Val or lie, preferably Leu, and/or
at position 442 where Asn (N) is substituted by Trp, Tyr, Phe or His, preferably Trp.

Preferably, the lacto-N-biosidase comprises a polypeptide sequence that has a sequence identity of at least 70% to the segment from amino acid positions 31 to 625 of SEQ ID No.1, and the following amino acid mutations, in which:
at position 410 Gly (G) is substituted by Trp, Tyr, Phe or His, preferably Trp; and/or
at position 416 Asp (D) is substituted by Asn or Gln, preferably Asn.

Also preferably, the lacto-N-biosidase comprises a polypeptide sequence that has a sequence identity of at least 75%, preferably at least 80%, more preferably at least 85%, yet more preferably at least 90%, still even more preferably at least 95% to the segment from amino acid positions 31 to 625 of SEQ ID No.1 and a mutation:
at position 410 where Gly (G) is substituted by Trp, Tyr, Phe or His, preferably Trp; and/or
at position 416 where Asp (D) is substituted by Asn or Gln, preferably Asn; and/or
at position 439 where Met (M) is substituted by Leu, Val or lie, preferably Leu, and/or
at position 442 where Asn (N) is substituted by Trp, Tyr, Phe or His, preferably Trp.

Preferably, the lacto-N-biosidase of this first aspect comprises a polypeptide sequence that has a sequence identity of at least 75%, preferably at least 80%, more preferably at least 85%, yet more preferably at least 90%, still even more preferably at least 95% to the segment from amino acid positions 31 to 625 of SEQ ID No.1, and the following amino acid mutations, in which:
at position 410 Gly (G) is substituted by Trp, Tyr, Phe or His, preferably Trp; and/or
at position 416 Asp (D) is substituted by Asn or Gln, preferably Asn.

More preferably, the lacto-N-biosidase comprises a polypeptide sequence that is identical with the segment from amino acid positions 31 to 625 of SEQ ID No.1 and a mutation:
at position 410 where Gly (G) is substituted by Trp, Tyr, Phe or His, preferably Trp; and/or
at position 416 where Asp (D) is substituted by Asn or Gln, preferably Asn; and/or
at position 439 where Met (M) is substituted by Leu, Val or lie, preferably Leu, and/or
at position 442 where Asn (N) is substituted by Trp, Tyr, Phe or His, preferably Trp.

Even more preferably, the lacto-N-biosidase comprises a polypeptide sequence that identical with the segment from amino acid positions 31 to 625 of SEQ ID No.1, and the following amino acid mutations, in which:
at position 410 Gly (G) is substituted by Trp, Tyr, Phe or His, preferably Trp; and/or
at position 416 Asp (D) is substituted by Asn or Gln, preferably Asn.

The embodiments of the first aspect, including the preferred and more preferred embodiments, may be His-tagged at the N- or C-terminus. A His-tag is a short sequence of DNA that codes for a specific polypeptide, which is frequently inserted into a target gene at the point of coding for expression at either the N- or C-terminus of the protein required. The method of His-tagging is especially useful as it allows for easy purification and detection of the recombinant protein. In one embodiment, the His-tag is attached to the N-terminus. In other embodiment, the His-tag is attached to the C-terminus. The presence of a His-tag does not affect markedly the basic characteristics of the claimed artificial lacto-N-biosidase mutants.

According to the second aspect of the invention, a method is provided for making a mutated lacto-N-biosidase of the first aspect of the invention, comprising the steps of:
(a) providing a DNA sequence encoding the mutated lacto-N-biosidase, then
(b) expressing the mutated lacto-N-biosidase in a host cell transformed with the DNA sequence obtained in step (a).

Step (a) can be carried out in a conventional manner by making a mutant DNA sequence encoding the mutated lacto-N-biosidase of the first aspect of the invention. In step (b) the so-mutated DNA sequence is then introduced at the gene level by usual molecular-biological methods. The DNA sequence of the enzyme variants can be cloned in an expression vector which can be introduced in an appropriate host expression strain such as E. coli, containing DNA plasmids with the required information for regulation of expression of the enzyme variant. The sequence encoding the enzyme variant can be placed under the control of an inducible promoter. As a result, by adding an inducer, the expression of the enzyme variant can be controlled (generally, isopropyl-β-D-thiogalactopyranoside (IPTG) is used). The so-transformed host cells are then cultured in conventional nutrient media (e.g. Lennox broth, minimal medium M9) and induced with IPTG. After expression, the biomass can be harvested by centrifugation. The mutated enzyme can be isolated from the biomass after appropriate cell lysis and purification. In this process, conventional centrifugation, precipitation, ultrafiltration and/or chromatographic methods can be used.

According to the third aspect of the invention, a method is provided for synthesizing a lacto-N-biose containing carbohydrate by reacting a lacto-N-biosyl donor and a carbohydrate acceptor in the presence of LnbX, its truncated functional analogs or a mutated lacto-N-biosidase according to the first aspect of the invention, whereby the lacto-N-biosyl residue of the lacto-N-biosyl donor is transferred to the carbohydrate acceptor.

The carbohydrate acceptor used in the third aspect of the invention can be any mono-, di- or oligosaccharide, preferably an oligosaccharide of 3-10 monosaccharide units, that has a terminal monosaccharide unit where the lacto-N-biosyl moiety can be transferred by LnbX, its truncated functional analogs or a mutated lacto-N-biosidase according to the first aspect of the invention. The oligosaccharide acceptor preferably contains a galactose unit at the non-reducing terminal. In one preferred embodiment, the galactose-containing acceptor is lactose. In other preferred embodiment, the acceptor comprises an N-acetyl-glucosamine unit adjacent to the galactose unit forming thus an N-acetyl-lactosaminyl (Galpβ1-4GlcNAcp) or a lacto-N-biosyl (Galpβ1-3GlcNAcp) moiety, preferably N-acetyl-lactosaminyl moiety. Examples of such acceptors are N-acetyl-lactosamine, LNnT (Galβ1-4GlcNAcβ1-3Galβ1-4Glc) or Galβ1-4GlcNAcβ1-3Galβ1-4GlcNAcβ1-3Galβ1-4Glc.

LnbX, its truncated functional analogs or a mutated lacto-N-biosidase according to the first aspect of the invention demonstrate a strong β1-3 selectivity when carrying out the method of the third aspect of the invention. As a result, the product of the reaction is an β1-3-lacto-N-biosyl mono-, di- or oligosaccharide, preferably an oligosaccharide of 3-10 monomer units. Preferably, LnbX, its truncated functional analogs or a mutated lacto-N-biosidase according to the first aspect of the invention bring the lacto-N-biosyl residue of an appropriate donor to the 3-position of the terminal galactose in an acceptor. Accordingly, LnbX, its truncated functional analogs or a mutated lacto-N-biosidase according to the first aspect of the invention are preferably used to synthesize lacto-N-biose containing oligosaccharides wherein the lacto-N-biose is attached to a galactose, preferably lacto-N-biose containing linear core HMOs such as the ones listed in Table 2 below.

TABLE 2

| acceptor | product |
|---|---|
| lactose | lacto-N-tetraose (LNT) |
| LNnT | para-lacto-N-hexaose (pLNH) |
| Galβ1-4GlcNAcβ1-3Galβ1-4GlcNAcβ1-3Galβ1-4Glc | para-lacto-N-octaose (pLNO) |

The lacto-N-biosyl donor used in the third aspect of the invention can be any lacto-N-biosyl compound from which LnbX, its truncated functional analogs or a mutated lacto-N-biosidase according to the first aspect of the invention are able to transfer the lacto-N-biosyl residue to the carbohydrate acceptor as described above. Suitably, the lacto-N-biosyl donor can be a compound of formula 1 or 2

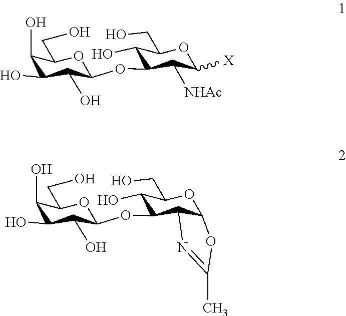

wherein X is selected from the group consisting of a mono-, di- or oligosaccharide, azide, fluoro, optionally substituted phenoxy, optionally substituted pyridinyloxy, group A, group B, group C and group D

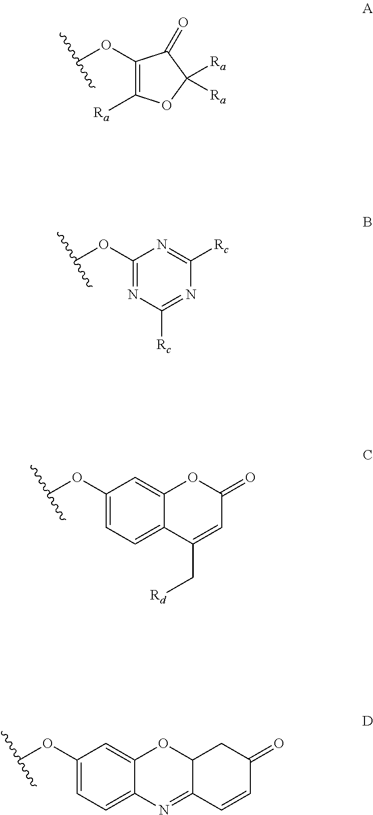

wherein Rₐ is independently H or alkyl, or two vicinal Rₐ groups represent a ═C(R_{b})₂ group, wherein R_{b} is independently H or alkyl, R_{c} is independently selected from the group consisting of alkoxy, amino, alkylamino and dialkylamino, R_{d} is selected from the group consisting of H, alkyl and —C(═O)Rₑ, wherein Rₑ is OH, alkoxy, amino, alkylamino, dialkylamino, hydrazino, alkylhydrazino, dialkylhydrazino or trialkylhydrazino, preferably X in formula 1 is selected from the group consisting of a mono-, di- or oligosaccharide, phenoxy-, p-nitrophenoxy-, 2,4-dinitrophenoxy- and 2-chloro-4-nitrophenoxy-group. Advantageously, X in formula 1 is lactose meaning that the lacto-N-biosyl donor is LNT.

It is beneficial if the method according to the third aspect of the invention is carried out with an acceptor/donor molar ratio 1:1 to 5:1, preferably 3:1 to 5:1, at a pH range of 4.5 to 6.5 and between room temperature and 60° C., preferably 35-55° C.

According to a fourth aspect of the invention, the use of LnbX, its truncated functional analogs or a mutated lacto-N-biosidase according to the first aspect of the invention is provided for synthesizing lacto-N-biose containing carbohydrates, preferably wherein the lacto-N-biose is attached to a terminal galactose, more preferably with β1-3 linkage to galactose, even more preferably lacto-N-biose containing linear core HMOs, particularly pLNH.

According to a fifth aspect of the invention, it is provided a mixture consisting or consisting essentially of LNT, LNnT, pLNH and optionally lactose.

According to a sixth aspect of the invention, it is provided a process for obtaining the mixture according to the fifth aspect of the invention, wherein said process comprises reacting LNT and LNnT in the presence of an enzyme selected from the group consisting of LnbX, its truncated functional analogs and a mutated lacto-N-biosidase according to the first aspect of the invention, to produce a reaction mixture, and then removing the enzyme and optionally lactose from the reaction mixture.

The mixture consisting or consisting essentially of LNT, LNnT, pLNH and optionally lactose can be formulated as a pharmaceutical, cosmetic and/or nutritional composition which may contain a pharmaceutically, cosmetic and/or nutritional acceptable carrier such as phosphate buffered saline solution, unbuffered saline solution, mixtures of ethanol in water, water and emulsions such as an oil/water or water/oil emulsion, as well as various wetting agents and/or excipients. The pharmaceutical, cosmetic and/or nutritional composition can also contain other materials that do not produce an adverse, allergic or otherwise unwanted reaction when administered to a patient.

Carriers and other materials comprised in the pharmaceutical, cosmetical and/or nutritional composition can include one or more of the following: solvents, dispersants, coatings, absorption promoting agents, controlled release agents and one or more inert excipients, such as starches, polyols, granulating agents, microcrystalline celluloses, diluents, lubricants, binders, and disintegrating agents. If desired, tablet dosages of the anti-infective mixtures can be coated by aqueous or non-aqueous techniques known to the skilled person.

A pharmaceutical, cosmetic and/or nutritional composition according to the present invention, can be administered orally, buccally, sublingually, topical and/or rectally. It can be formulated as a tablet, capsule, suppository, effervescent tablet, pellet, lozenge, troche, gel, paste, solution, suspension, emulsion, syrup, bolus, electuary, slurry, in an aqueous or non-aqueous liquid, as a powder, or as granules, containing a predetermined concentration of the mixture of HMOs.

Also, a pharmaceutical, cosmetic and/or nutritional composition according to the present invention can include binders, lubricants, inert diluents, flavouring agents, and humectants. A tablet, capsule, suppository or pellet containing the pharmaceutical, cosmetic and/or nutritional composition of the present invention can optionally be coated or formulated, so as to provide sustained, delayed or controlled release of the anti-infective mixture of HMOs.

The mixture of HMOs according to the invention may be supplemented with additional pharmaceutical agents, active pharmaceutical ingredients or agents having an effect to adverse health conditions of the patient whom the composition is administered.

In one embodiment, the composition can be in the form of a nutritional composition. For example, the nutritional composition can be a food composition, a rehydration solution, a medical food or food for special medical purposes, a nutritional supplement and the like. The nutritional composition can contain sources of protein, lipids and/or digestible carbohydrates and can be in powdered or liquid forms. The composition can be designed to be the sole source of nutrition or as a nutritional supplement.

Suitable protein sources include milk proteins, soy protein, rice protein, pea protein and oat protein, or mixtures thereof. Milk proteins can be in the form of milk protein concentrates, milk protein isolates, whey protein or casein, or mixtures of both. The protein can be whole protein or hydrolysed protein, either partially hydrolysed or extensively hydrolysed. Hydrolysed protein offers the advantage of easier digestion which can be important for humans with inflamed or compromised GI tracts. The protein can also be provided in the form of free amino acids. The protein can comprise about 5% to about 30% of the energy of the nutritional composition, normally about 10% to 20%. Ideally, the source of protein does not include excessive amounts of lactose.

The protein source can be a source of glutamine, threonine, cysteine, serine, proline, or a combination of these amino acids. The glutamine source can be a glutamine dipeptide and/or a glutamine enriched protein. Glutamine can be included due to the use of glutamine by enterocytes as an energy source. Threonine, serine and proline are important amino acids for the production of mucin. Mucin coats the GI tract and can improve intestinal barrier function and mucosal healing. Cysteine is a major precursor of glutathione, which is key for the antioxidant defences of the body.

Suitable digestible carbohydrates include maltodextrin, hydrolysed or modified starch or corn starch, glucose polymers, corn syrup, corn syrup solids, high fructose corn syrup, rice-derived carbohydrates, pea-derived carbohydrates, potato-derived carbohydrates, tapioca, sucrose, glucose, fructose, sucrose, honey, sugar alcohols (e.g. maltitol, erythritol, sorbitol), or mixtures thereof. Preferably the composition is reduced in or free from added lactose or other FODMAP carbohydrates. Generally digestible carbohydrates provide about 35% to about 55% of the energy of the nutritional composition. A suitable digestible carbohydrate is a low dextrose equivalent (DE) maltodextrin.

Suitable lipids include medium chain triglycerides (MCT) and long chain triglycerides (LCT). Preferably the lipid is a mixture of MCTs and LCTs. For example, MCTs can comprise about 30% to about 70% by weight of the lipids, more specifically about 50% to about 60% by weight. MCTs offer the advantage of easier digestion which can be important for humans with inflamed or compromised GI tracts. Generally, the lipids provide about 35% to about 50% of the energy of the nutritional composition. The lipids can contain essential fatty acids (omega-3 and omega-6 fatty acids). Preferably these polyunsaturated fatty acids provide less than about 30% of total energy of the lipid source.

Suitable sources of long chain triglycerides are rapeseed oil, sunflower seed oil, palm oil, soy oil, milk fat, corn oil, high oleic oils, and soy lecithin. Fractionated coconut oils are a suitable source of medium chain triglycerides. The lipid profile of the nutritional composition is preferably designed to have a polyunsaturated fatty acid omega-6 (n-6) to omega-3 (n-3) ratio of about 4:1 to about 10:1. For example, the n-6 to n-3 fatty acid ratio can be about 6:1 to about 9:1.

The nutritional composition may also include vitamins and minerals. If the nutritional composition is intended to be a sole source of nutrition, it preferably includes a complete vitamin and mineral profile. Examples of vitamins include vitamins A, B-complex (such as B1, B2, B6 and B12), C, D, E and K, niacin and acid vitamins such as pantothenic acid, folic acid and biotin. Examples of minerals include calcium, iron, zinc, magnesium, iodine, copper, phosphorus, manganese, potassium, chromium, molybdenum, selenium, nickel, tin, silicon, vanadium and boron.

The nutritional composition can also include a carotenoid such as lutein, lycopene, zeaxanthin, and beta-carotene. The total amount of carotenoid included can vary from about 0.001 μg/ml to about 10 μg/ml. Lutein can be included in an amount of from about 0.001 μg/ml to about 10 μg/ml, preferably from about 0.044 μg/ml to about 5 μg/ml of lutein. Lycopene can be included in an amount from about 0.001 μg/ml to about 10 μg/ml, preferably about 0.0185 μg/ml to about 5 μg/ml of lycopene. Beta-carotene can comprise from about 0.001 μg/ml to about 10 mg/ml, for example about 0.034 μg/ml to about 5 μg/ml of beta-carotene.

The nutritional composition preferably also contains reduced concentrations of sodium; for example, from about 300 mg/I to about 400 mg/I. The remaining electrolytes can be present in concentrations set to meet needs without providing an undue renal solute burden on kidney function. For example, potassium is preferably present in a range of about 1180 to about 1300 mg/I; and chloride is preferably present in a range of about 680 to about 800 mg/I.

The nutritional composition can also contain various other conventional ingredients such as preservatives, emulsifying agents, thickening agents, buffers, fibres and prebiotics (e.g. fructooligosaccharides, galactooligosaccharides), probiotics (e.g. B. animalis subsp. lactis BB-12, B. lactis HN019, B. lactis Bi07, B. infantis ATCC 15697, L. rhamnosus GG, L. rhamnosus HNOOI, L. acidophilus LA-5, L. acidophilus NCFM, L. fermentum CECT5716, B. longum BB536, B. longum AH1205, B. longum AH1206, B. breve M-16V, L. reuteri ATCC 55730, L. reuteri ATCC PTA-6485, L. reuteri DSM 17938), antioxidant/anti-inflammatory compounds including tocopherols, carotenoids, ascorbate/vitamin C, ascorbyl palmitate, polyphenols, glutathione, and superoxide dismutase (melon), other bioactive factors (e.g. growth hormones, cytokines, TFG-β), colorants, flavours, and stabilisers, lubricants, and so forth.

The nutritional composition can be formulated as a soluble powder, a liquid concentrate, or a ready-to-use formulation. The composition can be fed to a human in need via a nasogastric tube or orally. Various flavours and other additives can also be present.

The nutritional compositions can be prepared by any commonly used manufacturing techniques for preparing nutritional compositions in solid or liquid form. For example, the composition can be prepared by combining various feed solutions. A protein-in-fat feed solution can be prepared by heating and mixing the lipid source and then adding an emulsifier (e.g. lecithin), fat soluble vitamins, and at least a portion of the protein source while heating and stirring. A carbohydrate feed solution is then prepared by adding minerals, trace and ultra-trace minerals, thickening or suspending agents to water while heating and stirring. The resulting solution is held for 10 minutes with continued heat and agitation before adding carbohydrates (e.g. the HMOs and digestible carbohydrate sources). The resulting feed solutions are then blended together while heating and agitating and the pH adjusted to 6.6-7.0, after which the composition is subjected to high-temperature short-time processing during which the composition is heat treated, emulsified and homogenized, and then allowed to cool. Water soluble vitamins and ascorbic acid are added, the pH is adjusted to the desired range if necessary, flavours are added, and water is added to achieve the desired total solid level.

For a liquid product, the resulting solution can then be aseptically packed to form an aseptically packaged nutritional composition. In this form, the nutritional composition can be in ready-to-feed or concentrated liquid form. Alternatively, the composition can be spray-dried and processed and packaged as a reconstitutable powder.

When the nutritional product is a ready-to-feed nutritional liquid, it may be preferred that the total concentration of HMOs in the liquid, by weight of the liquid, is from about 0.1% to about 1.5%, including from about 0.2% to about 1.0%, for example from about 0.3% to about 0.7%. When the nutritional product is a concentrated nutritional liquid, it may be preferred that the total concentration of HMOs in the liquid, by weight of the liquid, is from about 0.2% to about 3.0%, including from about 0.4% to about 2.0%, for example from about 0.6% to about 1.5%.

In another embodiment, the nutritional composition is in a unit dosage form. The unit dosage form can contain an acceptable food-grade carrier, e.g. phosphate buffered saline solution, mixtures of ethanol in water, water and emulsions such as an oil/water or water/oil emulsion, as well as various wetting agents or excipients. The unit dosage form can also contain other materials that do not produce an adverse, allergic or otherwise unwanted reaction when administered to a human. The carriers and other materials can include solvents, dispersants, coatings, absorption promoting agents, controlled release agents, and one or more inert excipients, such as starches, polyols, granulating agents, microcrystalline cellulose, diluents, lubricants, binders, and disintegrating agents. Preferably the unit dosage form comprises primarily HMOs with a minimum amount of binders and/or excipients. Unit dosage forms are particularly suitable when nutritionally incomplete or not intended as a sole source of nutrition.

A unit dosage form can be administered orally, e.g. as a tablet, capsule, or pellet containing a predetermined amount of the mixture, or as a powder or granules containing a predetermined concentration of the mixture or a gel, paste, solution, suspension, emulsion, syrup, bolus, electuary, or slurry, in an aqueous or non-aqueous liquid, containing a predetermined concentration of the mixture. An orally administered composition can include one or more binders, lubricants, inert diluents, flavouring agents, and humectants. An orally administered composition such as a tablet can optionally be coated and can be formulated to provide sustained, delayed or controlled release of the HMOs.

A unit dosage form can also be administered by naso-gastric tube or direct infusion into the GI tract or stomach.

A unit dosage form can also include therapeutic agents such as antibiotics, probiotics, analgesics, and anti-inflammatory agents.

The proper dosage of a nutritional composition for a human can be determined in a conventional manner, based upon factors such as the concentration of HMOs, the human's condition, immune status, body weight and age. The required amount of HMOs would generally be in the range from about 1 g to about 15 g per day, in certain embodiments from about 2 g to about 10 g per day, for example about 3 g to about 7 g per day. Appropriate dose regimes can be determined by methods known to those skilled in the art.

In further embodiment, the mixture of HMOs can be formulated as a pharmaceutical composition. The pharmaceutical composition can contain a pharmaceutically acceptable carrier, e.g. phosphate buffered saline solution, mixtures of ethanol in water, water and emulsions such as an oil/water or water/oil emulsion, as well as various wetting agents or excipients. The pharmaceutical composition can also contain other materials that do not produce an adverse, allergic or otherwise unwanted reaction when administered to a human. The carriers and other materials can include solvents, dispersants, coatings, absorption promoting agents, controlled release agents, and one or more inert excipients, such as starches, polyols, granulating agents, microcrystalline cellulose, diluents, lubricants, binders, and disintegrating agents.

The pharmaceutical compositions can be administered orally, e.g. as a tablet, capsule, or pellet containing a predetermined amount, or as a powder or granules containing a predetermined concentration or a gel, paste, solution, suspension, emulsion, syrup, bolus, electuary, or slurry, in an aqueous or non-aqueous liquid, containing a predetermined concentration. Orally administered compositions can include binders, lubricants, inert diluents, flavouring agents, and humectants. Orally administered compositions such as tablets can optionally be coated and can be formulated to provide sustained, delayed or controlled release of the mixture therein.

The pharmaceutical compositions can also be administered by rectal suppository, aerosol tube, naso-gastric tube or direct infusion into the GI tract or stomach.

The pharmaceutical compositions can also include therapeutic agents such as antibiotics, probiotics, analgesics, and anti-inflammatory agents. The proper dosage of a pharmaceutical composition can be determined in a conventional manner, based upon factors such the concentration of the HMOs, the patient's condition, immune status, body weight and age. The required amount of HMOs would generally be in the range from about 1 g to about 15 g per day, in certain embodiments from about 2 g to about 10 g per day, for example about 3 g to about 7 g per day. Appropriate dose regimes can be determined by methods known to those skilled in the art.

The present invention also relates to the use of a mixture consisting of or consisting essentially of LNT, LNnT, pLNH and optionally lactose for medical purposes.

Individual HMOs are known to have beneficial health related effects and are known to affect bacterial and viral infections. While the isolated individual HMOs are beneficial in themselves, combinatorial mixtures of HMOs, as described herein, have a synergistic effect in the treatment of infections, compared to the isolated individual components.

Another aspect of the invention relates to a mixture consisting of or consisting essentially of LNT, LNnT, pLNH and optionally lactose, or a composition comprising the mixture consisting of or consisting essentially of LNT, LNnT, pLNH and optionally lactose, for use in treating, preventing and/or ameliorating a disease and/or a condition related to microbiome imbalance. Alternatively, one aspect of the invention relates to a method for treating, preventing and/or ameliorating a disease and/or a condition related to microbiome imbalance in a human comprising administering the human a mixture consisting of or consisting essentially of LNT, LNnT, pLNH and optionally lactose or a composition comprising the mixture consisting of or consisting essentially of LNT, LNnT, pLNH and optionally lactose. Typically, a mixture of LNT, LNnT and pLNH according to the present invention can be used for modulation of the microbiome of a human, e.g. to increase Bifidobacterium abundance and Barnesiella abundance, said modulation reduces Firmicutes abundance, especially Clostridia.

Furthermore, another aspect of the invention relates to a mixture consisting of or consisting essentially of LNT, LNnT, pLNH and optionally lactose, or a composition comprising the mixture consisting of or consisting essentially of LNT, LNnT, pLNH and optionally lactose, for use in treating and/or reducing the risk of a broad range of bacterial or viral infections in a human. Alternatively, one aspect of the invention relates to a method for treating and/or reducing the risk of a broad range of bacterial or viral infections in a human comprising administering the human a mixture consisting of or consisting essentially of LNT, LNnT, pLNH and optionally lactose or a composition comprising the mixture consisting of or consisting essentially of LNT, LNnT, pLNH and optionally lactose. Infections can arise in several body parts, not only limited to the gut microbiome, but also in other parts of the body exposed to the external environment, such as skin, hair, ears, eyes, nose and the respiratory system. One or more HMOs of the mixture according to the present invention can inhibit the adhesion of pathogenic bacteria, such as Pseudomonas aeruginosa or Campylobacter jejuni, uropathogenic and enteropathogenic Escherichia coli, certain Salmonella species and/or the pneumonia-causing pathogen Pseudomonas aeruginosa, as well as viruses such as norovirus. One or more HMOs of the mixture according to the present invention can also bind directly to pathogenic toxins, such as toxins from Clostridium difficile. One or more HMOs of the mixture according to the present invention may serve as an immune system modulator and affect intestinal health through stimulation of bifidobacteria. One or more HMOs of the mixture according to the present invention can inhibit the growth of Group B Streptococcus in both infants and breast milk. Group B Streptococcus are a leading cause of neonatal sepsis, pneumonia and meningitis. One or more HMOs of the mixture according to the present invention can bind directly to Shiga toxins Stx2 and Stx1B5 of Shigella dysenteriae. One or more HMOs of the mixture according to the present invention have the potential to reduce the risk of infectious diseases caused by either bacterial or viral pathogens most likely due to the binding to bacterial exotoxins.

Further, another aspect of the invention relates to a non-medical use of a mixture consisting of or consisting essentially of LNT, LNnT, pLNH and optionally lactose, or a composition comprising the mixture consisting of or consisting essentially of LNT, LNnT, pLNH and optionally lactose, for maintaining the commensal homeostasis of the gut microbiota in a human.

Further, another aspect of the invention relates to a non-medical use of a mixture consisting of or consisting essentially of LNT, LNnT, pLNH and optionally lactose, or a composition comprising the mixture consisting of or consisting essentially of LNT, LNnT, pLNH and optionally lactose, in the dietary management of a human. Dietary management means exclusive or partial feeding of patients who, because of a disease, disorder or medical condition are suffering from:
either have a limited, impaired or disturbed capacity to take, digest, absorb, metabolise or excrete ordinary food or certain nutrients contained therein, or metabolites, or
have other medically-determined nutrient requirements (see: Commission Notice on the classification of Food for Special Medical Purposes of the European Commission, Official Journal of the European Union C 401, 25.11.2017, p. 10-11).

The following numbered aspects of the invention are provided:

Aspect 1. A mutated lacto-N-biosidase having
an amino acid sequence that is at least 70% identical to the sequence from amino acid position 45 to 625 of SEQ ID No. 1, and
a mutation at least at one or more of amino acid positions selected from 410, 416, 439 and 442, said amino acid numbering being according to SEQ ID No. 1.

Aspect 2. The mutated lacto-N-biosidase according to aspect 1, comprising one or more of the following mutations:
at position 410 Gly (G) is substituted by Trp, Tyr, Phe or His, preferably Trp; and/or
at position 416 Asp (D) is substituted by Asn or Gln, preferably Asn; and/or
at position 439 Met (M) is substituted by Leu, Val or lie, preferably Leu; and/or
at position 442 Asn (N) is substituted by Trp, Tyr, Phe or His, preferably Trp.

Aspect 3. The mutated lacto-N-biosidase according to aspect 2, comprising a mutation:
at position 410 Gly (G) is substituted by Trp, Tyr, Phe or His, preferably Trp; and/or
at position 416 Asp (D) is substituted by Asn or Gln, preferably Asn.

Aspect 4. The mutated lacto-N-biosidase according to any of the precedent aspects, comprising or having a polypeptide sequence that has a sequence identity of at least 75%, preferably at least 80%, more preferably at least 85%, yet more preferably at least 90%, still even more preferably at least 95%, to the segment from amino acid position 45 to 625 of SEQ ID No.1.

Aspect 5. The mutated lacto-N-biosidase according to any of the precedent aspects, comprising or having a polypeptide sequence that is identical with the segment from amino acid positions 45 to 625 of SEQ ID No.1.

Aspect 6. The mutated lacto-N-biosidase according to any of the precedent aspects, comprising or having a polypeptide sequence that has a sequence identity of at least 70%, preferably at least 75%, more preferably at least 80%, even more preferably at least 85%, yet more preferably at least 90%, still even more preferably at least 95%, to the segment from amino acid position 31 to 625 of SEQ ID No.1.

Aspect 7. A method for making a mutated lacto-N-biosidase according to any of the aspects 1 to 6, comprising the steps of:
(a) providing a DNA sequence encoding the mutated lacto-N-biosidase, then
(b) expressing the mutated lacto-N-biosidase in a host cell transformed with the DNA sequence obtained in step (a).

Aspect 8. A method for synthesizing a lacto-N-biose containing carbohydrate by reacting a lacto-N-biosyl donor and a carbohydrate acceptor in the presence of an enzyme selected from the group consisting of LnbX, its truncated functional analogs and a mutated lacto-N-biosidase according to any of the aspects 1 to 6, whereby the lacto-N-biosyl residue of the lacto-N-biosyl donor is transferred to the carbohydrate acceptor, and wherein the lacto-N-biosyl donor is preferably a compound of formula 1 or 2

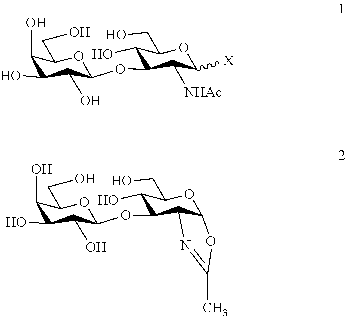

wherein X is selected from the group consisting of a mono-, di- or oligosaccharide, azide, fluoro, optionally substituted phenoxy, optionally substituted pyridinyloxy, group A, group B, group C and group D

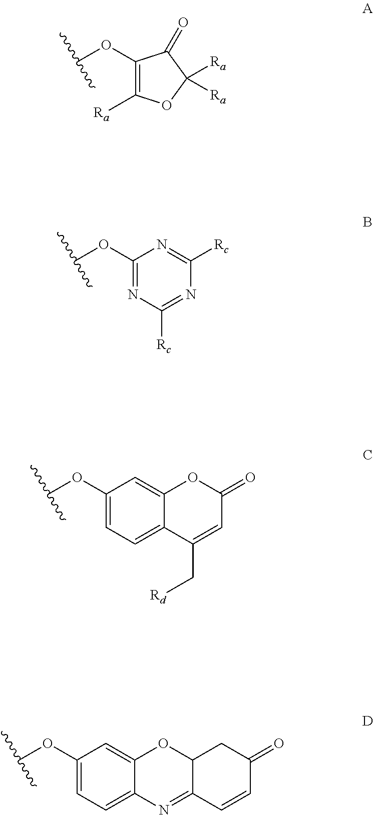

wherein Rₐ is independently H or alkyl, or two vicinal Rₐ groups represent a ═C(R_{b})₂ group, wherein R_{b} is independently H or alkyl, Rₑ is independently selected from the group consisting of alkoxy, amino, alkylamino and dialkylamino, R_{d} is selected from the group consisting of H, alkyl and —C(═O)Rₑ, wherein Rₑ is OH, alkoxy, amino, alkylamino, dialkylamino, hydrazino, alkylhydrazino, dialkylhydrazino or trialkylhydrazino,
more preferably X in formula 1 is selected from the group consisting of a mono-, di- or oligosaccharide, phenoxy-, p-nitrophenoxy-, 2,4-dinitrophenoxy- and 2-chloro-4-nitrophenoxy-group, advantageously the lacto-N-biosyl donor is LNT.

Aspect 9. The method according to aspect 8, wherein the donor is LNT, the acceptor is LNnT and the lacto-N-biose containing carbohydrate product is pLNH.

Aspect 10. A mixture consisting or consisting essentially of LNT, LNnT, pLNH and optionally lactose.

### EXAMPLES

In the examples below a truncated version of the lacto-N-biosidase from B. longum JCM1217 (LnbX, Sakamura et al. J. Biol. Chem. 288, 25194 (2013)), namely the segment from amino acid position 31 to 625 of SEQ ID No.1, together with its mutants were tested, the position of mutation is according to SEQ ID No. 1. The truncated LnbX (WT, wild type) and the mutants contained the His-tag MGSSHHHHHHSSGLVPRGSHM in the N-terminus.

### Example 1

The trans-lacto-N-biosidase activity of truncated LnbX and its mutants was investigated in the LNT+LNnT⇄pLNH+Lac reaction in which the formation of pLNH was followed. Starting conditions: LNT 10 mM, LNnT 100 mM, enzyme 0.25 μM, temperature 40° C., pH 6.0. The reactions were stopped by heating at 90° C. for 10 minutes after the time indicated below. Product formation was examined with HLPC. The structure of pLNH was confirmed with HPLC-MS and by means of combination of different NMR techniques.

HPLC conditions:

An Accucore-150-Amide-HILIC 2.6 μm column (150×3 mm) was used at 25° C. with a flow-rate of 0.8 ml/min using acetonitrile-water as mobile phase in gradient elution mode. Eluted substrates and products were detected with CAD. A quadratic calibration curve was used with its intercept forced to zero for the calculation of the concentration of each compound.

The results are summarized in the table below.

| variant | conversion (time) |
|---|---|
| WT | 18% (1 h) |
| D416N | 35% (1 h) |
| M439L | 23% (1 h) |
| G410W | 25% (3 h) |
| N442W | 18% (3 h) |

### Example 2

The reactions disclosed in Example 1 were carried out for extended reaction times. For WT, D416N, N442W, and M439L, all pLNH was hydrolysed within 24 h, whereas for G410W, 95% of the formed pLNH was hydrolysed within 24 h. The product hydrolysis was most rapid for WT. All variants showed a lower rate of product hydrolysis than the WT in the following order (from the higher to the lower): D416N, N442W, M439L, G410W. The variants showed higher transglycosylation/hydrolysis rate than WT in the following order (from the higher to the lower): D416N, M439L, G410W, N442W.

### Example 3

Further tests were carried out with mutant D416N to find optimal reaction conditions. The following variables were tested:
temperature: 35-55° C.,
pH: 4.5-6.5,
LNT concentration: 10-100 mM,
acceptor/donor molar ratio: 1-5,
enzyme concentration: 0.1-1 μM.

Under each condition (28 in total) samples were collected at the following times: 15 min, 30 min, 1 h, 3 h and 5 h.

The data showed that the effect of pH and temperature was marginal, higher LNT concentration and higher acceptor/donor ratio increased the yield of pLNH and the product purity (expressed in molar % of pLNH compared to the sum of hexa- and octasaccharides formed and detected). Based on the data obtained, an optimal condition is: acceptor/donor ratio 5:1, 0.55 μM enzyme, pH 4.5-6.5, temperature 35-55° C. Results of the best conditions are listed in the table below.

| temp | | LNT | LNnT | enzyme | | | pLNH |
|---|---|---|---|---|---|---|---|
| (° C.) | pH | (mM) | (mM) | (μM) | conversion | time | purity |
| 35 | 6.5 | 100 | 500 | 1 | 57% | 3 h | 82% |
| 55 | 4.5 | 100 | 500 | 0.55 | 52% | 1 h | 84% |
| 45 | 5.5 | 100 | 300 | 0.55 | 46% | 3 h | 78% |
| 55 | 6.5 | 100 | 500 | 0.1 | 30% | 5 h | 79% |
| 35 | 4.5 | 100 | 500 | 0.1 | 30% | 5 h | 81% |

1.-16. (canceled)

17. A mutated lacto-N-biosidase having
(a) an amino acid sequence that is at least 90% identical to the sequence from amino acid position 45 to 625 of SEQ ID NO: 1, and
(b) a mutation at least at one or more of amino acid positions 410, 416, 439, and 442 where the amino acid numbering is according to SEQ ID NO: 1,
wherein the mutation:
at position 410, Gly (G) is substituted with Trp, Tyr, Phe, or His, preferably Trp; and/or
at position 416, Asp (D) is substituted with Asn or Gln, preferably Asn; and/or
at position 439, Met (M) is substituted with Leu, Val, or Ile, preferably Leu; and/or
at position 442, Asn (N) is substituted with Trp, Tyr, Phe, or His, preferably Trp.

18. The mutated lacto-N-biosidase according to claim 17, wherein the mutation at position 410 is Gly (G) substituted with Trp.

19. The mutated lacto-N-biosidase according to claim 17, wherein the mutation at position 416 is Asp (D) substituted with Asn.

20. The mutated lacto-N-biosidase according to claim 17, wherein the mutation at position 439 is Met (M) substituted with Leu.

21. The mutated lacto-N-biosidase according to claim 17, wherein the mutation at position 442 is Asn (N) substituted with Trp.

22. The mutated lacto-N-biosidase according to claim 18, wherein the mutation at position 416 is Asp (D) substituted with Asn.

23. The mutated lacto-N-biosidase according to claim 22, wherein the mutation at position 439 is Met (M) substituted with Leu.

24. The mutated lacto-N-biosidase according to claim 23, wherein the mutation at position 442 is Asn (N) substituted with Trp.

25. The mutated lacto-N-biosidase according to claim 17, wherein the mutation consists of substitutions at positions 410 and 416.

26. The mutated lacto-N-biosidase according to claim 17, wherein the mutated lacto-N-biosidase comprises an amino acid sequence that has a sequence identity of at least 95% to the segment from amino acid position 45 to 625 of SEQ ID NO: 1.

27. The mutated lacto-N-biosidase according to claim 25, wherein the mutated lacto-N-biosidase comprises an amino acid sequence that has a sequence identity of at least 95% to the segment from amino acid position 45 to 625 of SEQ ID NO: 1.

28. The mutated lacto-N-biosidase according to claim 17, wherein the mutated lacto-N-biosidase comprises an amino acid sequence that is identical to the segment from amino acid positions 45 to 625 of SEQ ID NO: 1, except for one or more mutations at positions 410, 416, 439 and 442 where the amino acid numbering is according to SEQ ID NO: 1.

29. A method for making a mutated lacto-N-biosidase according to claim 17, comprising the steps of:
(a) providing a DNA sequence encoding the mutated lacto-N-biosidase, then
(b) expressing the mutated lacto-N-biosidase in a host cell transformed with the DNA sequence obtained in step (a).

30. A method for synthesizing a lacto-N-biose containing carbohydrate by reacting a lacto-N-biosyl donor and a carbohydrate acceptor in the presence of an enzyme selected from the group consisting of LnbX, a trucated functional analog of LnbX, and a mutated lacto-N-biosidase according to claim 17, whereby the lacto-N-biosyl residue of the lacto-N-biosyl donor is transferred to the carbohydrate acceptor.

31. The method according to claim 30, lacto-N-biosyl donor is a compound of formula 1 or 2 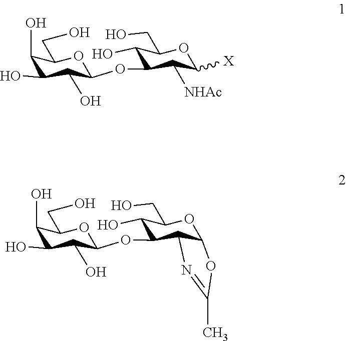
wherein is selected from the group consisting of a monosaccharide, disaccharide, oligosaccharide, azide, fluoro, optionally substituted phenoxy, optionally substituted pyridinyloxy, group A, group B, group C, and group D 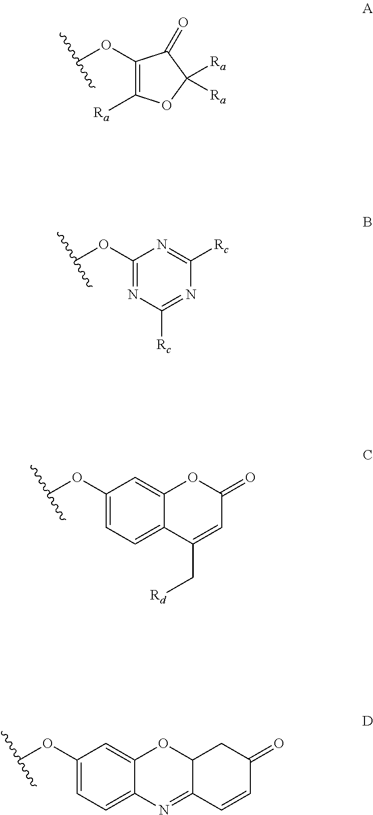
wherein Rₐ is independently H or alkyl, or two vicinal R1 groups represent a ═C(R_{b})₂ group, wherein R_{b} is independently H or alkyl, R_{c} is independently selected from the group consisting of alkoxy, amino, alkylamino, and dialkylamino, R_{d} is selected from the group consisting of H, alkyl and —C(═O)Re, wherein Rₑ is OH, alkoxy, amino, alkylamino, dialkylamino, hydrazino, alkylhydrazino, dialkylhydrazino, or trialkylhydrazino.

32. The method according to claim 30, wherein X in formula 1 is selected from the group consisting of a monosaccharide; disaccharide; oligosaccharide; phenoxy-; p-nitrophenoxy-; 2,4-dinitrophenoxy- and 2-chloro-4-notrophenoxy-group.

33. The method according to claim 32, wherein the lacto-N-biosidase is lacto-N-tetraose (LNT).

34. The method according to claim 33, wherein the acceptor is lacto-N-neotetraose (LNnT).

35. The method according to claim 34, wherein the lacto-N-biose containing carbohydrate product is para-lacto-N-hexaose (pLNH).


SEQUENCE LISTING (1 sequence)

SEQ ID NO: 1 - protein, 1599 aa, Bifidobacterium longum
MTSRQGRQAIAATAAMGVAVALALPTAAFAQSATQGKETATTTSSGTTYYVSSAHGDDANAGTSENAPWKSLTKVNDIASDLGPGDSVLLEYGSEFNDQYLHIKDTAGNADAPITISAYGDADEGKPVIASNGVKGSQWEQDYRANVGNHKNKGTVSTTLLLKDVSYITVSNLEITNDDADVYDPIDTWKWTDTPDSDGTKLDRSASRMDRTGVAGIAENGATMSNVTLDNLYIHDVDGNIYNKHMANGGIYFMAHYPMENTSAETDVWLREHVSRFDHVTIRNSTVKDVDRWGIAVGYTAYLNYIDANYGDGSIDDALIAKYGSTNVRIENNYVKGAGGDAITLMYCDRPVIEHNVGDSVSKHINTQDYTQPGSYGGRVAAGIWPWRCKDPVFQYNEMYNNLNAEHGNGDGQAWDADYGDGTLYQYNYSYGNSFASLMICNWYAVNTTFRYNISQNDRQGVFDLPSNGPGNHIYNNTVYVDADSQVLTKRSNSQSLFENNIFINATNTKKTETWNRGSQNGGQTYDNNMYVNYANKPTSDANAIEADDVSAVLAGAGSAPTSALKSGAEHARTGEKAAFDGYRPVAGSKAINAGKVVSDLNDYAVENDFLGNAVKGRPDLGAVESDVVSVTMASSKYETGTETDSGTGDKTKVIHVTFTDKNPVTVKELLSNVSADKGVDKAVYRVADAKSGKSADARSAESEPNMLDRLLSLLPGSDRNAKDDETKLADSEPVRDGDILRFSAEGTDETDEYTIRQRITWDWVADYEQGVADFDWKAQRRTSAGGEWTTISAYDGSWPNTVYDQYYGVGVNGTLAELSGDRKQTHGLLIDKPGDGLPTAMAWKAPESGTVMLSLKTFADKIAEPYLRQNADNAGKKVTLSLMRNDETLCSADDLSVYQKSSEQFAQCLAEHGSIDVQEGDWIRIVADAETGVKAPSLHISPVITYEDKAPAAPKQNVRYDVSYAATDAVVGTQSAVAAAFTADGGEADAPDGVAFAFKDGGDEGEASPVIDASTGAVTFTPAAGQYGATVTRTVVVTYADGSSDETTVTFRVAQSHAQRLNVLYPTVRGDAGTDLKRTPKFTLKADGAAASVPEGTTFALGANAPAGASVDMANGTVTLNSGVGGTVTVPVTVTFADDGASVSSTARFEVTAPAALGSSELETATVDGVNVVYAPFSADSPMTVAQLLAKVTAEPSGADKGVYRDGVRLEAGAELAENDVLRFSAKGSTVSDDYVVKSKTTWDWVNDFQVRVQGPIWYGQRQTEADGVWSDIADFDATYPNWMYETYYGPGVDYANHSLPTDRSAIHGLISDSPASAGGSAMAWKAPKAGTVKVSIREDEPYLRQDGSNGKALTLRLMHDDKVVCFADLTVSKQRSEEFANCVADKGEIAVEAGDWIRVTATSASGMNKPSAHISPVIAYMAASTPGPEPVPVDKSTLKATVEEALGLAESDYTDESWAALVAARDAAQTVLDDDAATAEQVETAQNALRDAIDGLEKKPVDPDPNPKPDPNPDPDPTPDPDPDPGPDTKPGDGSGNGSGTGNGSGSGNGSTGSGSDGATTGGKLTATGADVAGAAAMVALTAAAGIGLAAAARRRR